(19) United States
(12) Patent Application Publication
(10) Pub. No.: US 2008/0214869 A1
(43) Pub. Date: Sep. 4, 2008

(54) Processes for purification of tigecycline

(76) Inventors: Sergei Fine, Qiriat-Arbaa (IL); Slavik Yurkovski, Kiryat-Gat (IL); Sofia Gorohovsky-Rosenberg, Be'er Sheva (IL); Evgeny Tsiperman, Be'er Sheva (IL)

Correspondence Address: KENYON & KENYON LLP, ONE BROADWAY, NEW YORK, NY, 10004, US

(21) Appl. No.: 12/074,453

(22) Filed: Mar. 3, 2008

Related U.S. Application Data
(60) Provisional application No. 60/904,532, filed on Mar. 1, 2007.
(60) Provisional application No. 60/925,022, filed on Apr. 18, 2007.

(51) Int. Cl. C07C 235/84

(52) U.S. Cl. 564/163

(57) ABSTRACT

The invention is directed to improved processes of purifying tigecycline.

### CROSS-REFERENCE TO RELATED APPLICATIONS

This application claims the benefit of U.S. provisional application Ser. Nos. 60/904,532, filed Mar. 1, 2007, and 60/925,022, filed Apr. 18, 2007, hereby incorporated by reference.

### FIELD OF THE INVENTION

The invention is directed to improved processes of purifying tigecycline.

### BACKGROUND

Tigecycline (CAS 220620-09-7), (4S,4aS,5aR,12aS)-9-(2-(tert-butylamino) acetamido)-4,7-bis(dimethylamino)-1,4,4a,5,5a,6,11,12a-octahydro-3,10,12,12a-tetrahydroxy-1,11-dioxo-2-naphthacenecarboxamide is the first drug of a new generation of tetracycline antibiotics called glycylcyclines. Tigecycline has a wider range of bioactivity than the parent tetracycline and its analogues discovered so far, and may be administered less frequently and/or in lower doses.

Tigecycline has been introduced and marketed by Wyeth under the brand name TYGACIL® and it is especially indicated against acute lethal infections caused by Gram-negative bacteria. TYGACIL® is marketed as leophilized powder or cake for intravenous injection and the drug substance does not contain excipients or preservatives.

Tigecycline has the following structure:

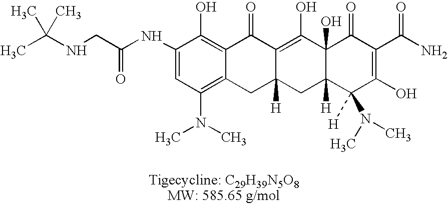

and is described in U.S. Pat. Nos. 5,494,903 and 5,284,963.

Tetracyclines, in general, and tigecycline specifically, are very sensitive to various factors such as acidity, exposure to light and heat, etc. which may cause relatively rapid degradation that result in formation of numerous impurities like oxidation and hydrolysis products, such as, a C4-epimer of the compound. U.S. Pat. No. 5,248,797 discloses precipitation of Tigecycline in diethyl ether. U.S. Pat. No. 5,675,030 apparently reports purifying tigecycline by extraction using dichloromethane, a polar aprotic solvent. International Published Application No. WO 2006/130431 apparently reports obtaining tigecycline with reduced amount of the C-4 epimer by use of a polar protic or a mixture of polar protic solvents and aprotic solvents.

However, there still exists a need in the art for additional and improved means of purifying crude tigecycline without increasing the amount of the C-4 epimer.

### SUMMARY OF THE INVENTION

In one embodiment, the present invention encompasses a process for preparing tigecycline having a purity of at least about 98.5% or containing less than about 1.5% of its C-4 epimer.

In one embodiment, the present invention encompasses a method of purifying tigecycline by a process comprising exposing solid tigecycline to one or more non-polar aprotic solvent, wherein no organic protic solvent is used.

In another embodiment of the present invention, there is provided a method of purifying tigecycline by a process comprising treating tigecycline with one or more non-polar aprotic solvents, or water or a mixture thereof, wherein no organic protic solvent is used.

In another embodiment, the present invention encompasses a purified tigecycline prepared by a process comprising treating tigecycline with one or more non-polar aprotic solvents, or water or a mixture thereof, wherein no organic protic solvent is used, and the purified tigecycline has a purity of at least about 98.5% pure or contains less than about 1.5% of its C-4 epimer.

### DETAILED DESCRIPTION OF THE INVENTION

The present invention provides a more simple method of purifying Tigecycline purification is achieved without the need for precipitation nor extraction.

The present invention provides a method of purifying (crude) tigecycline, purified tigecycline and pharmaceutical compositions thereof.

As used herein, unless otherwise defined, “%” refers to weight percent (% w/w) relating to the weight of one component to the total weight of the composition.

In one embodiment, the present invention encompasses a method of purifying tigecycline by a process comprising admixing tigecycline with an non-polar aprotic solvent, wherein no organic protic solvent is used. The process comprises providing (crude) tigecycline and admixing it with a non-polar aprotic solvent for a period of time to obtain a purified tigecycline.

In another embodiment of the present invention, there is provided a method of purifying tigecycline by a process comprising treating tigecycline with one or more non-polar aprotic solvent, or water or a mixture thereof, wherein no organic protic solvent is used. The process comprises providing (crude) tigecycline and admixing it with one or more non-polar aprotic solvents or water or mixtures thereof for a period of time to obtain a purified tigecycline.

Non-polar aprotic solvents used in the present invention are selected from the group consisting of: C₆₋₇ aromatic and C₅₋₇ aliphatic hydrocarbons, C₃₋₄ alkoxy, C₃₋₈ ethers, C₂₋₆ esters of acids, C₃₋₈ ketones, C₂₋₄ nitriles, C₂₋₃ amides, C₃₋₅ organic carbonates and mixtures thereof.

Preferably, the C₆₋₇ aromatic hydrocarbons are benzene or toluene. C₅₋₇ aliphatic hydrocarbons can be linear or branched. Preferably, the C₅₋₇ aliphatic hydrocarbons are n-pentane, n-hexane or n-heptane. Preferably, the C₃₄ alkoxy are dimethoxymethane or dimethoxyethane. Preferably, the C₃₋₈ ethers are diethyl ether, tetrahydrofuran (“THF”), methyl tetrahydrofuran, or cyclopentyl methyl ether. Preferably, the C₂₋₆ esters of acids are methyl acetate, ethyl acetate, isobutyl acetate or butyl acetate. Preferably, the C₃₋₈ ketones are acetone or methyl isobutyl ketone. Preferably, the C₂₋₄ nitriles are acetonitrile or butyronitrile. Preferably, the C₂₋₃ amides are acetamide or dimethylformamide (“DMF”) Preferably, the C₃₋₅ organic carbonates are dimethyl carbonate or ethyl carbonate.

Most preferably, the non-polar aprotic solvents are n-heptane, toluene, dimethoxyethane, ethyl acetate, THF, acetone, acetonitrile or dimethyl carbonate.

Optionally, water can be added with one or more aprotic solvents.

The starting (crude) tigecycline may be in solid or semisolid form.

The admixing step of the purification process may be performed at temperatures of about −20° C. to about 120° C., preferably at about −10° C. to about 40° C. and, more preferably, at about 0° C. to about 25° C. Further, the time period for which tigecycline is admixed with the solvent or mixture of solvents sufficient to obtain the purified tigecycline may be carried out at said temperature for a period of about 15 minutes to about 4 hours, preferably for about 30 minutes to about 2 hours, more preferably for about 30 minutes to about 1 hour depending on the temperature and the amount of tigecycline. Purified tigecycline can then be isolated using any method known to the person skilled in the art, for example, filtration or centrifugation.

The obtained purified tigecycline has a purity of at least about 98.5%, more preferably of about 98.5% to about 99.5% pure. Typically, the obtained purified tigecycline contains less than about 1.5% of its C-4 epimer. Preferably, the purified tigecycline contains less than about 1% of its C-4 epimer, more preferably, the purified tigecycline contains less than about 0.5% of its C-4 epimer. The C-4 epimer has the following structure:

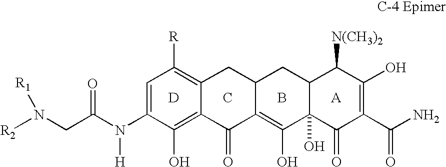

Having described the invention with reference to certain preferred embodiments, other embodiments will become apparent to one skilled in the art from consideration of the specification. The invention is further defined by reference with the following examples describing in detail the purification of tigecycline. It will be apparent to those skilled in the art that many modifications, both to materials and methods, may be practiced without departing from the scope of the invention.

### EXAMPLES

### Example 1

### Purification of Tigecycline in a Single Aprotic Solvent

Tigecycline (2 g) characterized by chromatographic purity of 97.3%, as measured by HPLC area %, was stirred in 20 ml of ethyl acetate at 0-5° C. for 1 hour, whereupon the solid was collected by means of vacuum filtration, washed with cold ethyl acetate and air-dried. Tigecycline thus obtained was characterized by chromatographic purity of 98.57%, as measured by HPLC area %.

### Example 2

### Purification of Tigecycline with a Mixture of Aprotic Solvents

Tigecycline (5 g) characterized by chromatographic purity of 98.17%, as measured by HPLC area %, was mixed with 50 ml of water and pH of the mixture was adjusted at 5. The resulted solution was extracted once with dichloromethane and pH of the aqueous phase was adjusted at 7, whereupon it was extracted with dichloromethane seven times. The combined DCM organic extract phase was washed with water, dried over sodium sulfate and then mixed with 100 ml of ethyl acetate. The resulted solution of DCM-ethylacetate was concentrated to about 50 ml and the residual suspension was stirred for half an hour at 0-5° C. The solid was then collected by means of vacuum filtration, washed with cold ethyl acetate, air-dried and, finally, dried under vacuum at 40° C. thus affording tigecycline characterized by chromatographic purity of 99.26%, as measured by HPLC area %.

What is claimed is:

1. A method of purifying tigecycline comprising providing tigecycline and admixing tigecycline with one or more non-polar aprotic solvents to obtain a purified tigecycline, wherein no organic protic solvent is used.

2. The method of claim 1, wherein the non-polar aprotic solvent is selected from the group consisting of: C₆₋₇ aromatic and C₅₋₇ aliphatic hydrocarbons, C₃₄ alkoxy, C₃₋₈ ethers, C₂₋₆ esters of acids, C₃₋₈ ketones, C₂₋₄ nitriles, C₂₋₃ amides, C₃₋₅ organic carbonates and mixtures thereof.

3. The method of claim 2, wherein the non-polar aprotic solvent is n-heptane, toluene, dimethoxyethane, ethyl acetate, THF, acetone, acetonitrile or dimethyl carbonate.

4. The method of claim 1, wherein admixing tigecycline with one or more non-polar aprotic solvents is performed at a temperature of about −20° C. to about 120° C.

5. The method of claim 1, wherein admixing tigecycline with one or more non-polar aprotic solvents is carried out for a period of about 15 minutes to about 4 hours.

6. The method of claim 1, wherein the purified tigecycline obtained has a purity of at least about 98.5% weight to weight.

7. The method of claim 1, wherein the purified tigecycline obtained contains less than about 1.5% of its C-4 epimer.

8. The method of claim 7, wherein the purified tigecycline obtained contains less than about 1% of its C-4 epimer.
